(19) United States
(12) Patent Application Publication
(10) Pub. No.: US 2007/0055201 A1
(43) Pub. Date: Mar. 8, 2007

(54) Systems and methods for providing cavities in interior body regions

(76) Inventors: Christine L. Seto, Los Angeles, CA (US); Paul M. Sand, San Carlos, CA (US); Meera L. Sankaran, Cupertino, CA (US); Warren Sapida, Santa Clara, CA (US)

Correspondence Address: KILPATRICK STOCKTON LLP - 55461, 1001 WEST FOURTH STREET, WINSTON-SALEM, NC, 27101, US

(21) Appl. No.: 11/483,464

(22) Filed: Jul. 10, 2006

Related U.S. Application Data
(60) Provisional application No. 60/698,287, filed on Jul. 11, 2005.

(51) Int. Cl. A61M 29/00

(52) U.S. Cl. 604/96.01

(57) ABSTRACT

Systems and methods for providing a cavity in an interior body region are described. In one described method, a frame comprising a plurality of frame members disposed adjacent an expansible body is inserted into a treatment area along a path established by a hollow member. The expansible body is expanded in the treatment area, and the expansible body is constrained from expanding by the frame.

### CROSS-REFERENCE TO RELATED APPLICATIONS

The present application for patent claims priority to U.S. Provisional Patent Application Ser. No. 60/698,287, filed Jul. 11, 2005 and entitled “Systems and Methods for Providing Cavities in Interior Body Regions,” the disclosure of which is hereby incorporated in full by reference.

### FIELD OF THE INVENTION

The invention relates to systems and methods for providing cavities in interior body regions for diagnostic or therapeutic purposes.

### BACKGROUND

Certain diagnostic or therapeutic procedures require provision of a cavity in an interior body region. For example, as disclosed in U.S. Pat. Nos. 4,969,888 and 5,108,404, which are incorporated herein by reference, an expansible body may be deployed to form a cavity in cancellous bone tissue, as part of a therapeutic procedure that fixes fractures or other abnormal bone conditions, both osteoporotic and non-osteoporotic in origin. The expansible body or other expansible body may compress the cancellous bone to form an interior cavity. The cavity may receive a filler material, such as a bone cement, which provides renewed interior structural support for cortical bone.

This procedure can be used to treat cortical bone, which due to osteoporosis, avascular necrosis, cancer, trauma, or other disease is fractured or is prone to compression fracture or collapse. These conditions, if not successfully treated, can result in deformities, chronic complications, and an overall adverse impact upon the quality of life. However, as an expansible body is expanded during such a procedure, it may not expand to a shape and dimension desired by a user of the device.

A demand exists for further systems and methods that are capable of providing cavities in bone and other interior body regions in safe and efficacious ways.

### SUMMARY

Embodiments of the present invention provide systems and methods for providing cavities in interior body regions. One illustrative embodiment comprises an expansible body configured to be deployed within a treatment area, and a frame comprising a plurality of frame members disposed adjacent the expansible body. The frame may be configured to constrain expansion of the expansible body, and to be maneuverable along a path established by a hollow member.

This embodiment is mentioned not to limit or define the invention, but to provide an example of an embodiment of the invention to aid understanding thereof. Illustrative embodiments are discussed in the Detailed Description, and further description of the invention is provided there. Advantages offered by the various embodiments of the present invention may be further understood by examining this specification.

### BRIEF DESCRIPTION OF THE FIGURES

These and other features, aspects, and advantages of the present invention are better understood when the following Detailed Description is read with reference to the accompanying drawings, wherein:

FIG. 1 is a perspective view of a tool according to one embodiment of the present invention;

FIG. 2 is an enlarged end view of the tool shown in FIG. 1, wherein the frame thereof is shown in a contracted state;

FIG. 3 is an enlarged end view of the tool shown in FIG. 1, wherein the frame thereof is shown in an expanded state;

FIG. 4 is a close-up perspective view of a tool according to one embodiment of the present invention;

FIG. 5 is an elevation (lateral) view of several human vertebrae, with a hollow member establishing a percutaneous path to a vertebral body of one of the several vertebrae;

FIG. 6 is a plan (coronal) view of a human vertebra being accessed by a hollow member, with portions removed to reveal cancellous bone within a vertebral body;

FIG. 7 is an elevation (lateral) view of a human vertebra comprising a vertical compression fracture condition and with a tool according to one embodiment of the present invention deployed to provide a cavity within a vertebral body;

FIG. 8 is an elevation (lateral) view of the tool and vertebra of FIG. 7, wherein the expansible body is shown in an expanded state, and the vertebral body is shown with an increased internal dimension resulting from the expansion of the expansible body constrained by the adjacent frame;

FIG. 9 is a plan (coronal) view of a tool comprising an edge configured to shear tissue according to one embodiment of the present invention, wherein the tool is shown being rotated in cancellous bone;

FIG. 10 is a flow chart of a method according to one embodiment of the present invention;

FIG. 11 is a plan view of a sterile kit configured to store a single use tool according to one embodiment of the present invention; and

FIG. 12 is an exploded perspective view of the sterile kit of FIG. 11.

### DETAILED DESCRIPTION

Embodiments of the present invention provide systems and methods for providing cavities in interior body regions. The systems and methods embodying the invention can be adapted for use in many suitable interior body regions, wherever the formation of a cavity within or adjacent one or more layers of tissue may be required for a therapeutic or diagnostic purpose. The illustrative embodiments show the invention in association with systems and methods used to treat bones. In other embodiments, the present invention may be used in other interior body regions or types of tissues.

As used in this specification and the appended claims, the singular forms “a,” “an,” and “the” include plural referents unless the context clearly dictates otherwise. Thus, for example, the term “a lumen” is intended to mean a single lumen or a combination of lumens.

Furthermore, the words “proximal” and “distal” refer to directions closer to and away from, respectively, an operator (e.g., surgeon, physician, nurse, technician, etc.). An operator may insert a medical device into a patient, with at least a tip-end (i.e., distal end) of the device inserted inside a patient's body. Thus, in one example, the end of the medical device inserted inside the patient's body would be the distal end of the medical device, while the end of the medical device outside the patient's body would be the proximal end of the medical device. In another example, the entire medical device may be inserted inside the patient's body, where the distal end of the medical device may extend further inside the patient's body than the proximal end of the medical device.

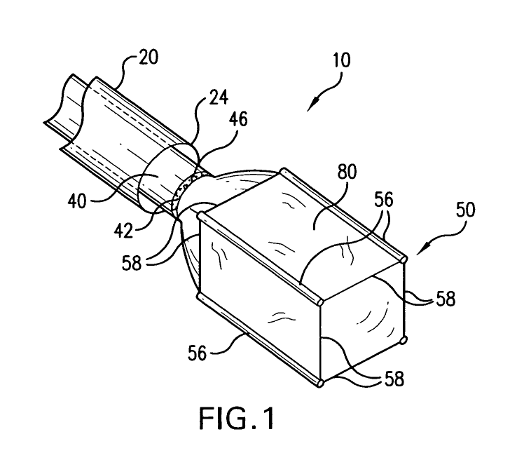

Referring now to the Figures, in which like part numbers depict like elements throughout the Figures, FIG. 1 is a perspective view of a system 10 according to one embodiment of the present invention. The system 10 shown in FIG. 1 is configured to allow an operator to provide a cavity in a targeted treatment area. The system 10 is further configured to be used in a kyphoplasty procedure to restore height to a vertebra suffering from a vertical compression fracture condition.

The system 10 comprises a hollow member 20 comprising a proximal end (not shown) and a distal end 24. The hollow member 20 may comprise, for example, a flexible or inflexible cannula, and may be fabricated from a material selected to facilitate advancement and rotation of an elongate member 40 maneuverable along a path established by the hollow member 20. For example, the elongate member 40 may be movably disposed within the hollow member 20. The hollow member 20 can be constructed, for example, using standard flexible, medical grade plastic materials, such as vinyl, nylon, polyethylenes, ionomer, polyurethane, and polyethylene tetraphthalate (PET). At least some portion of the hollow member 20 can also comprise more rigid materials to impart greater stiffness and thereby aid in its manipulation and torque transmission capabilities. More rigid materials that can be used for this purpose comprise stainless steel, nickel-titanium alloys (such as Nitinol), and other metal alloys.

The system 10 shown in FIG. 1 further comprises the elongate member 40 configured to be maneuverable along the path established by the hollow member 20. The elongate member 40 may be made from a resilient inert material providing torsion transmission capabilities (e.g., stainless steel, a nickel-titanium alloy such as Nitinol, and other suitable metal alloys). In other embodiments, the elongate member 40 may be fashioned from a variety of suitable materials, comprising a carbon fiber, a glass, or a flexible material, such as a plastic or rubber. In one embodiment comprising a flexible elongate member 40, the elongate member 40 may be, for example, fashioned from twisted wire filaments, such stainless steel, nickel-titanium alloys (such as Nitinol), and suitable other metal alloys.

The elongate member 40 shown is hollow, allowing movement of a flowable material within a bore therethrough along its axis. A flowable material may comprise, for example, a liquid material, a gaseous material, a slurry, a sludge, a plasma, a paste, a flowable solid (such as powdered, pulverized, granulated, pelletized, or encapsulated material), or any other suitable material that may flow naturally or be made to flow from one place to another. The elongate member 40 shown comprises a fitting 42 at its distal end 46. The elongate member 40 may comprise a handle (not shown) at its proximal end (not shown) to aid in gripping and maneuvering the elongate member 40. For example, in one embodiment, such a handle can be made of a foam material secured about the proximal end elongate member 40.

The system 10 shown in FIG. 1, further comprises a frame 50 configured to be maneuverable along the path established by the hollow member 20. For example, the frame 50 may be movably disposed within the hollow member 20. The frame 50 is configured to slide and rotate within the hollow member 20. The system 10 further comprises an expansible body 80 configured to be deployed in a targeted treatment area along a path established by the hollow member 20. For example, the expansible body 80 may be percutaneously deployed within cancellous bone tissue in a vertebral body. The expansible body 80 is disposed at the distal end 46 of the elongate member 40. In the embodiment shown, the expansible body 80 is disposed within the frame 50. In a different embodiment, at least a portion of the frame 50 may be disposed within the expansible body 80.

The expansible body 80 shown comprises a single aperture that is coupled to the fitting 42 at the distal end 46 of the elongate member 40. Movement of a flowable material through the hollow elongate member 40 and the aperture into the interior of the expansible body 80 may expand the expansible body 80. The expansible body 80 may be contracted by movement of a flowable material out of the expansible body 80 through the aperture and the bore through the hollow elongate member 40.

The frame 50 is also disposed at the distal end 46 of the elongate member 40. The frame 50 is configured to be maneuverable along a path established by the hollow member 20. For example, in one embodiment, the hollow member 20 may be inserted, and then removed, leaving a path through a plurality of tissue layers to the treatment area along which the frame 50 may be maneuvered. In another embodiment, the hollow member 20 may remain in place during a procedure, and the frame 50 may be movably disposed within the hollow member 20. For example, the frame 50 may be slid or rotated while within the hollow member 20.

The frame 50 is disposed adjacent the expansible body 80. In one variation, the frame 50 is configured to constrain expansion of the expansible body 80 as the expansible body 80 expands. In the embodiment shown, the expansible body 80 is configured to be expanded, and the frame 50 is configured to constrain the expansion of the expansible body, once the frame 50 and the expansible body 80 have been inserted through the hollow member 20 to a point beyond the distal end 24 of the hollow member 20 as shown in FIG. 1.

In some embodiments, at least a portion of the frame 50 may be fashioned from a shape memory alloy, and may be configured to at least partly expand in a treatment area beyond the distal end 24 of the hollow member 20 without assistance from expansible body 80. In one such embodiment, the expansible body 80 may be configured to be inserted into the frame 50 once it has been at least partly expanded in a treatment area. In another embodiment, at least a portion of the frame 50 may be coupled to the expansible body 80.

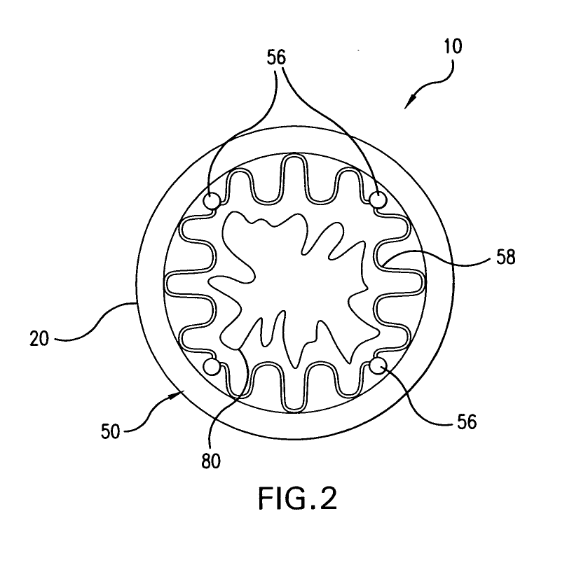

In the embodiment shown in FIG. 1, the frame 50 is further configured to collapse as the expansible body 80 contracts. The contracted expansible body 80 and the collapsed frame 50 (see FIG. 2) may then be withdrawn into the distal end 24 of the hollow member 20. In another embodiment, the frame 50 may be configured to, at least temporarily, maintain at least one expanded dimension when the expansible body 80 contracts. For example, the frame 50 may be expansible and fashioned from a shape memory material (e.g., Nitinol), and the expansible body 80 may be configured to be temporarily disposed in the frame 50, and removed after the frame 50 has been expanded.

In the embodiment shown in FIG. 1, the frame 50 comprises a plurality of elongate rigid frame members 56, each coupled at both ends to two pliable frame members 58. In the embodiment shown, the rigid frame members 56 are fashioned from a surgical grade stainless steel, and the pliable frame members 58 are fashioned from a polymer. In another embodiment, at least a portion of the frame 50, such as at least one of the rigid frame members 56 or at least one of the pliable frame members 58, may be fashioned from a surgical grade shape memory material formed with a resilient memory, such as a nickel titanium alloy. In another embodiment, at least a portion of the frame 50 may be fashioned from a plastic, a latex, or any other suitable material. For example, in one such embodiment, at least a portion of the frame 50 may be fashioned from the same material as the expansible body 80 is fashioned from, and the expansible body 80 and frame 50 may be coupled and co-extruded.

In the embodiment shown, each of the four rigid frame members 56 is axially oriented about the expansible body 80 along the axis of the elongate member 40. The proximal and distal ends of each of the rigid frame members 57 are coupled to the proximal and distal ends, respectively, of the two adjacent rigid frame members 56 via the pliable frame members 58, forming a rectangular box-shaped structure when the expansible body 80 is expanded as shown. The rigid frame members 56 may be coupled to the pliable frame members 58 through the use of welding, gluing, bonding, melting, or any other suitable fastener (such as a screw, a rivet, a tack, a staple, a nail, etc.). In another embodiment, the entire frame 50 may be fashioned from the same material, and may be injection molded, cast, forged, or machined.

Each of the plurality of rigid frame members 56 shown in FIG. 1 comprises a round cross section. In other embodiments, one or more of the frame members 56 can, in cross section, be polygonal, rectilinear, ovoid, asymmetrical, or any other suitable configuration.

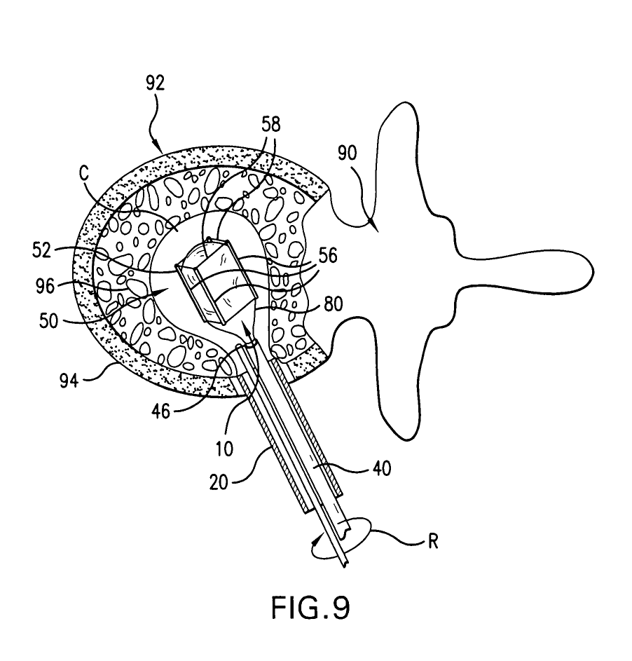

In another embodiment (see FIG. 9), each of the plurality of rigid frame members 56 may comprise at least one edge that is configured to contact and shear (curette) tissue when the expansible body 80 is expanded. Such an edge may comprise, for example, a beveled edge, a deburred edge, a serrated edge, a blade tip, or a raised edge. In one such embodiment, while the expansible body 80 is expanded and constrained by the frame 50, the elongate member 40 may be rotated in the hollow member 20, thereby rotating at least one of the frame 50 and the expansible body 80. In yet another embodiment, another portion of the frame 50, or the elongate member 40 may comprise a surface configured to directly contact and shear at least one layer of tissue.

In an embodiment comprising an edge configured to contact and shear tissue, the frame 50 may be configured to cut adjacent tissue mass in the targeted treatment area when rotated, providing or enlarging a cavity in the tissue. In another embodiment, the proximal end of at least one of the hollow member 20 and the elongate member 40 may carry a fitting (not shown) that, in use, may be coupled to an electric motor (not shown). The motor may thus rotate one or both of the elongate member 40 and the hollow member 20, thereby rotating the frame 50.

In another embodiment of the present invention, at least a portion of the frame 50 or the elongate member 40 may comprise one or more radiological markers. For example, in the embodiment shown in FIGS. 1-3, one or more of the plurality of rigid frame members 56 may comprise one or more radiological markers. The markers may be fashioned from a radiopaque material, such as platinum, gold, calcium, tantalum, and other heavy metals.

In an embodiment employing a plurality of radiological markers, a first set of markers may be placed at or near a proximal end of the rigid frame members 56, while another set of markers may be placed at a location on the frame 50 spaced apart from the first marker, such as at a point at or near the distal end of each of the rigid frame members 56. In another embodiment, the distal end 46 of the elongate member 40, or the distal end 24 of the hollow member 20 can carry one or more markers. A radiological marker may permit radiologic visualization of at least one of the elongate member 40, the frame 50, and the hollow member 20 within a targeted treatment area. In other embodiments, other forms of markers can be used to allow a user to visualize the location, size, and shape of at least the frame 50 or the expansible body 80 within the targeted treatment area. For example, the expansible body may be expanded with a radiopaque gas or liquid.

A tool according to one embodiment of the present invention, such as the system 10 described with respect to FIGS. 1-3, can comprise an interior lumen. The lumen may be coupled to an external source of fluid and an external vacuum source. In one such embodiment, a rinsing liquid, e.g., sterile saline, can be introduced from the source through the lumen into the targeted tissue region before, during or after the system 10 provides a cavity in a tissue mass. The rinsing liquid may reduce friction and conduct heat away from the tissue during a cutting operation. The rinsing liquid can be introduced continuously or intermittently while the tissue mass is being compacted, removed, or cut. The rinsing liquid can also carry an anticoagulant or other anti-clotting agent. In one such embodiment, the lumen may be coupled to the vacuum source, and liquids and debris can be aspirated from the targeted tissue region through the lumen.

In yet another embodiment of the present invention, a sheath may surround at least one of the frame 50 and the expansible body 80. In one such embodiment, the frame 50 may be disposed between the sheath and the expansible body 80, and the sheath may be coupled to the expansible body 80. For example, the sheath may be fabricated from a substantially non-compliant and rupture-resistant material, such as Mylar or a suitable plastic. In another embodiment, a sheath may be fabricated from a compliant material, such as latex. In another embodiment, a sheath may be coupled to the inside of the frame 50. A sheath according to one embodiment may hold one or more frame members, such as the plurality of rigid frame members 56, in a desired arrangement, thereby constraining the expansion of the expansible body 80. A sheath may also prevent dislodged tissue mass from puncturing the expansible body 80 or becoming caught within the frame 50.

Referring now to FIG. 2, an enlarged end view of the system 10 shown in FIG. 1 is shown, wherein the expansible body 80 is shown in a contracted state at a point within the hollow member 20. While in the contracted state shown in FIG. 2, the expansible body 80 comprises a first dimension. As shown in FIG. 2, while the expansible body 80 is in the contracted state, the adjacent frame 50, also fits within the inside bore dimension of the hollow member 20, thereby providing a clearance. Such a clearance may allow a user of the system 10 to maneuver the expansible body 80, the frame 50 and the elongate member 40 within and along the axis of the hollow member 20. In other embodiments, at least one of the expansible body 80, the frame 50, and the elongate member 40 may be configured to be rotated with respect to the hollow member 20.

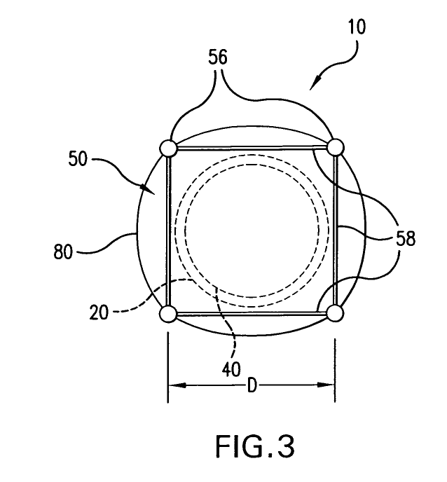

Referring now to FIG. 3, an enlarged end view of the system 10 is shown, wherein the expansible body 80 is shown in an expanded state. While in the expanded state shown in FIG. 3, the expansible body 80 comprises a second dimension. The expansible body 80 is configured to be expanded from the first dimension shown in FIG. 2 to the second dimension shown in FIG. 3 when extended to a point beyond the distal end 24 of the hollow member 20. The expansible body 80 is further configured to be contracted from the second dimension shown in FIG. 3 to the first dimension shown in FIG. 2 prior to or when brought within the distal end 24 of the hollow member 20 from a point beyond the distal end 24 of the hollow member 20.

As shown in FIG. 3, when expanded, the expansible body 80 is constrained from further expansion by the fully expanded frame 50. The expansible body 80, as shown in FIG. 3, is constrained to a substantially rectangular-shaped cross section (as viewed in FIG. 3) by the adjacent frame 50. In other embodiments, the expansible body 80 may be constrained to a different shape, based, at least in part, on the total number, configuration, or orientation of the frame members 56. For example, in one embodiment, a frame 50 may constrain an expansible body 80 to a substantially octagon-shaped cross section. The frame 50, in the embodiment shown in FIG. 3, comprises a filly expanded dimension, D, as measured perpendicular to the axis of the elongate member 40. In the embodiment shown in FIG. 3, the fully expanded dimension, D, is larger than the inside diameter of the interior bore of the hollow member 20.

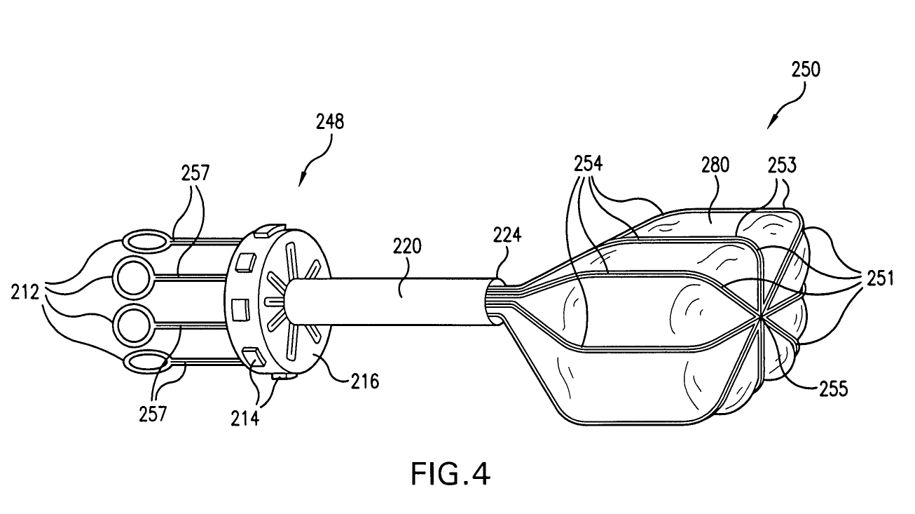

Referring now to FIG. 4, a perspective view of a tool 210 according to one embodiment of the present invention is shown. As shown in FIG. 4, the tool 210 comprises an expansible body 280 and a frame 250 disposed adjacent the expansible body 280. The expansible body 280 and the adjacent frame 250 have been extended beyond a distal end of a hollow member 220. The hollow member 220, may comprise, for example, a flexible cannula. The expansible body 280 shown is expanded with a material (such as a radiopaque material as described above) through an aperture (not shown) facing the distal end 224 of the hollow member 220.

The frame 250 shown in FIG. 4 comprises a plurality of frame members 254. Each of the plurality of frame members 254 comprises a structural member 251 and a tubular member 253. The structural members 251 shown comprise a metal wire filament. The tubular members 253 shown comprise a plastic tubing material. The plurality of tubular members 253 are adjacent the expansible body 280, and each encase a corresponding structural member 251.

The frame members 254 in the embodiment shown are coupled to each other at their distal ends 255. In other embodiments comprising a plurality of frame members 254, at least one of the plurality of frame members 254 may not be coupled to any other frame member 254, or may comprise a continuous loop of material constraining the expansible body 280. The tubular members 253 of the frame members 254 in the embodiment shown in FIG. 4 are further coupled to the expansible body 280 by a bonding process. The tubular members 253 in the pictured embodiment comprise a material that is more compatible with the material from which the expansible body 280 is fabricated than the material from which the structural members 251 are fabricated, facilitating coupling with the expansible body 280. In other embodiments, one or more of the tubular members 253 may not be coupled to the expansible body 280, or one or more of the structural members 251 may be coupled directly to the expansible body 280.

The device 210 shown in FIG. 4 further comprises a controller 248. The controller 248 is configured to allow an operator of the device 210 to individually adjust the tension in each of the plurality of frame members 254, thereby constraining the expansible body 280 by adjusting a corresponding dimension of the expansible body 280. Each of the plurality of frame members 254 passes through a hollow member 220 and is coupled at its proximal end 257 to a finger loop 212.

The finger loops 212 coupled to a frame members 254 may be used by an operator of the tool 210 to increase or decrease the tension in the attached frame member 254 by pulling or pushing, respectively, the finger loop 212 while simultaneously pushing a button 214 on a guide 216. In the embodiment shown, the buttons 214 are coupled to a spring within the guide 216 (not shown). In the embodiment shown, only when a button 214 is pushed can the tension in one of the frame members 254 be adjusted by pulling or pushing one of the finger loops 212.

In other embodiments, at least some portion of the frame 250 may be in communication with a different type of controller, such as a slide controller, a pistol grip controller, a ratcheting controller, a threaded controller, or any other suitable type of controller that can be configured to permit an operator of the device 210 to control at least one of the extent to which the frame 250 extends beyond a distal end 224 of the hollow member 220, and the extent to which the frame 250 is constraining expansion of the expansible body 280 or controlling a dimension thereof. In one embodiment of the present invention, the controller 248 can also comprise indicia by which the physician can visually estimate at least one of the extent to which the expansible body 280 is expanded, the extent to which the frame 250 is extended beyond the distal end 224 of the hollow member 220, and the extent to which an expanded dimension of the expansible body 280 has been adjusted.

In the embodiment shown in FIG. 4, the device 210 may be used to provide a cavity in an interior body region. A user of the device 210 may be able to use the controller 248 to adjust the size and shape of the expansible body 280 within the cavity. For example, by individually adjusting the tension in the frame members 254 using the controller 248, the user may be able to adjust a plurality of different dimensions of the expansible body 280, forcing the expansible body 280 to expand and provide force to surrounding tissues to provide a cavity of desired shape and dimension.

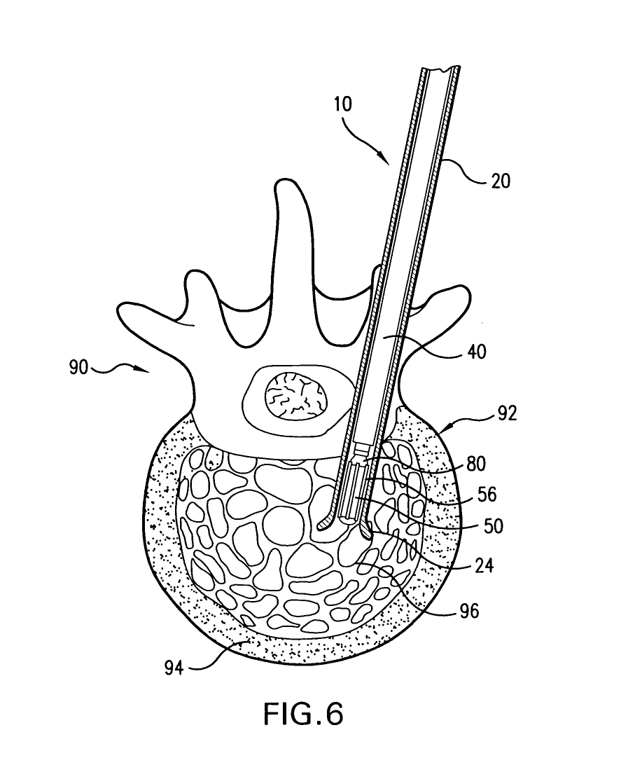

Upon provision of such a cavity, the expansible body 280 may be contracted. The frame 250 and the expansible body 280 may then be removed from the interior body region through the hollow member 220. Once removed, a material, such as a bone cement (e.g., polymethylmethacrylate (PMMA) bone cement), may then be used to fill the cavity provided by the tool 210. Such an embodiment may be useful in situations where the tool 210 is used to restore height to a vertebral body (see FIGS. 6-9). The bone cement may be inserted, either via the hollow member 220, or via a separate hollow member (such as a contralateral hollow member).

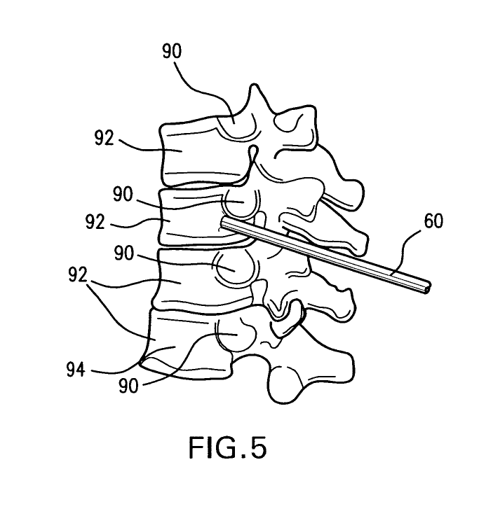

Referring now to FIG. 5, an elevation (lateral) view of several human vertebrae 90 is shown, with a hollow member 20 establishing a percutaneous path along its axis to a vertebral body 92 of one of the several vertebrae. The vertebral body 92 extends on the anterior (i.e., front or chest) side of the vertebra 90. The vertebral body 92 comprises an exterior formed from compact cortical bone 94. The cortical bone 94 encloses an interior volume of reticulated cancellous, or spongy, bone 96 (also called medullary bone or trabecular bone-shown in FIGS. 6-9).

The vertebral body 92 is in the shape of an oval disc. As FIGS. 5-9 show, access to the interior volume of the vertebral body 92 can be achieved, e.g., by drilling an access portal through a rear side of the vertebral body 92, (a postero-lateral approach). The portal for the postero-lateral approach enters at a posterior side of the vertebral body 92 and extends anteriorly into the vertebral body 92. The portal can be provided either with a closed, minimally invasive procedure or with an open procedure.

Alternatively, access into the interior volume can be accomplished by drilling an access portal through one or both pedicles of the vertebra 90. This is called a transpedicular approach. Access into the interior of the vertebral body may also be accomplished using an extrapedicular approach alongside a pedicle of the vertebra 90, or anteriorly. It is the physician who ultimately decides which access site is indicated.

A tool according to the present invention may be configured to be deployed within a treatment area adjacent at least one layer of tissue by movement within and along a path formed by the axis of the hollow member 20. For example, as shown in FIG. 5, the hollow member 20 may provide a tool, such as the tools 10 or 210 described above, with access to the cancellous bone within the vertebral body 92 of a vertebra 90 to provide a cavity therewithin. Such a cavity may be provided during a procedure for restoring some of the height of a vertebral body lost due to a vertical compression fracture or other pathology or trauma, prior to insertion of a filler material, such as a bone cement, into the vertebral body 92.

It should be appreciated, however, that systems and methods according to the present invention are not limited in application to human vertebrae, and may be used to provide cavities within or curette other parts of a living or non-living organism. For example, the system 10 can be deployed in other embodiments in other bone types and within or adjacent other tissue types, such as in a vertebral disc, a knee joint, etc.

Referring now to FIG. 6, a plan (coronal) view of a vertebra 90 being accessed by a system according to one embodiment of the present invention. The vertebra 90 is shown, with portions removed to reveal cancellous bone 96 within the vertebral body 92. As shown in FIG. 6, the system 10 as described above with respect to FIGS. 1-3 has been inserted into the hollow member 20 for access to the vertebral body 92 of the vertebra 90. The hollow member 20 is shown with portions removed to reveal the elongate member 40, the expansible body 80, and the frame 50 therewithin. The system 10 is configured to be maneuverable along a path established by the axis of the hollow member 20.

In the embodiment shown in FIG. 6, once beyond a distal end 24 of the hollow member 20, the expansible body 80 may be expanded from a contracted state (see FIG. 2) to an expanded state (see FIG. 3), to provide a cavity within the cancellous bone 96.

In use, the elongate member 40 may be substantially carried for sliding and rotation within the hollow member 20. The user of the system 10 may freely slide the elongate member 40 axially within the hollow member 20 to deploy the expansible body 80 and the adjacent frame 50 in a targeted treatment site. When deployed at the site, the user can extend the expansible body 80 and the adjacent frame 50 to a point beyond the distal end 24 of the hollow member 20 adjacent cancellous bone tissue 96 within the vertebral body 92. The user may also able to rotate the elongate member 40 within the hollow member 20 and thereby the expansible body 80 and the frame 50 to adjust at least one of their orientation and travel path.

In one embodiment, at least a portion of the frame 50 may be fabricated from a shape-memory alloy. In such an embodiment, at least a portion of the frame 50 may comprise a tendency to spring open to assume a preset, native expanded dimension once beyond the distal end 24 of the hollow member 20. In another embodiment, the frame 50 may be adjustable. For example, the frame 50 may comprise a plurality of frame members, each individually adjustable to control at least one dimension of the expansible body 80 (see FIG. 4). Using such an embodiment, an operator of the system 10 may use a controller (such as the controller 248, described above) to alter the size and shape of the expansible body 80 by adjusting one or more of the plurality of adjustable frame members.

In other embodiments of the present invention, one or both of a spring member and a screw member may be coupled to the frame 50. The spring member or the screw member may be configured to expand or contract the frame 50. For example, a screw member may be in communication with a controller (such as the controller 248, described above) to allow an operator of the system 10 to control expansion or contraction of the frame 50. In yet another embodiment, a spring member may provide a spring force configured to assist or resist expansion or contraction of the frame 50.

In the embodiment shown in FIG. 6, once the frame 50 adjacent the expansible body 80 is beyond the distal end 24 of the hollow member 20, the user of the system 10 may also rotate the elongate member 40, thereby rotating the frame 50 and expansible body 80 disposed at the distal end 46 of the elongate member 40. As discussed below with respect to FIG. 9, rotation of the frame 50 may slice or cut through surrounding tissue mass, in this case, cancellous bone 96.

In other embodiments, at least one of the frame 50, the expansible body 80, the elongate member 40, and the hollow member 20 can carry one or more radiological markers, as previously described. The markers may allow radiologic visualization of the frame 50 and the expansible body 80 and their positions relative to the hollow member 20 and the vertebra 90 while in use within a targeted treatment area.

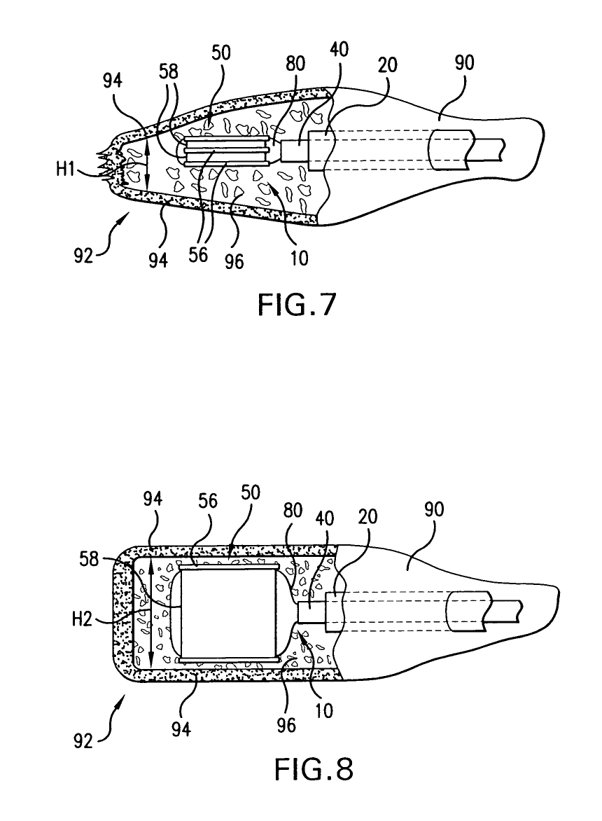

Referring now to FIG. 7, an elevation (lateral) view of a human vertebra 90 comprising a vertical compression fracture condition is shown. As shown in FIG. 7, a vertebral body 92 of the vertebra 90 has been partially crushed due to an osteoporotic condition of cancellous bone 96 therewithin. The dimension H1 of the vertebral body 92 has been decreased as a result of this fracture. A hollow member 20 has been percutaneously inserted to provide access to the cancellous bone 96 within the vertebral body 92.

In the embodiment shown in FIG. 7, the elongate member 40, the expansible body 80, and the frame 50 of the system 10 described with respect to FIGS. 1-3 have been inserted into a treatment area within the vertebral body 94 through the hollow member 20 in a contracted state (as shown in FIG. 2). The user of the system 10 may wish to use it to provide a cavity within the vertebral body 92, and to restore height to the vertebral body 92 lost when the fracture occurred.

Referring now to FIG. 8, an elevation (lateral) view of the human vertebra 90 of FIG. 7 is shown after the system 10 has increased the height of the vertebral body 92 to dimension H2 from dimension H1 as shown in FIG. 7. As shown in FIG. 8, the frame 50 and the expansible body 80 disposed at the distal end 46 of the elongate member 40 have been expanded to a fully expanded state (as shown in FIG. 3) as a result of the inflation and resultant constrained expansion of the expansible body 80. Such an increase in the dimension H2 may allow a physician using the system 10 to at least partially restore the vertebra 90 to a shape analogous to its pre-vertical compression fracture condition.

Referring now to FIG. 9, a plan (coronal) view of the system 10 and the vertebra 90 shown in FIGS. 7 and 8 is shown, wherein the system 10 is shown being rotated in the cancellous bone 96 of the vertebral body 92. As shown in FIG. 9, the frame 50 comprises a surface 52 configured to contact and shear cancellous bone tissue when the frame 50 has been expanded. The elongate member 40 may be rotated (as indicated by arrow R) within the hollow member 20, thereby rotating the expansible body 80 and the frame 50 disposed at the distal end 46 of the elongate member 40. In one such embodiment, once expanded, the surface 52 of the frame 50 may act as a curette when the elongate member 40 is rotated by a user through surrounding cancellous bone 96 (as indicated by arrow R in FIG. 9). Accordingly, the rotating frame 50 may cut cancellous bone 96 in the vertebral body 92, enlarging or changing the proportions or dimensions of a cavity provided therein. In the embodiment shown in FIG. 9, the surface 52 is configured to directly contact and shear the cancellous bone 96 to help a user of the system 10 to provide a cavity C of a desired shape and dimension.

In one embodiment, a suction tube may also be deployed through the hollow member 20 to remove cancellous bone cut by the surface 52. In yet another embodiment, the system may comprise an interior lumen to serve as a suction tube as well as to convey a rinsing liquid into the cavity as it is being formed. The suction tube (or a lumen) may introduce a rinsing fluid (with an anticoagulant, if desired) and may remove cancellous bone cut by the surface 52. Alternatively, the hollow member 20 may comprise a first interior lumen that serves as a suction tube, and a second interior lumen that serves to flush the treatment area. In one embodiment, by periodically inflating or deflating the expansible body 80, thereby expanding or collapsing, respectively, the frame 50, or by rotating the elongate member 40, a user of the system 10 may provide a cavity C having the desired dimensions.

Once the desired cavity C is formed, the cavity-providing tool, such as system 10, may be withdrawn through the hollow member 20. In one embodiment, the cavity C may then be at least partially filled with a filler material, such as a bone cement, or another suitable tool can then be deployed through the hollow member 20, or through another hollow member (such as a contralateral hollow member) into the formed cavity C. A second tool can, for example, perform a diagnostic or therapeutic procedure (such as filling the cavity C with a bone cement). In other embodiments other materials (such as a therapeutic material) may be provided into the cavity C by at least one of the expansible body 80 and the frame 50 while it is deployed in the vertebral body 92. For example, an allograft material, a synthetic bone substitute, a medication, or a flowable material that may set to a hardened condition may be provided into the cavity C. The procedure may also be used to apply radiation therapy or chemotherapy. Further details of the injection of such materials into the cavity C for therapeutic purposes may be found in U.S. Pat. Nos. 4,969,888 and 5,108,404, and in co-pending U.S. patent application Publication No. 2003/0229372, which are incorporated herein by reference.

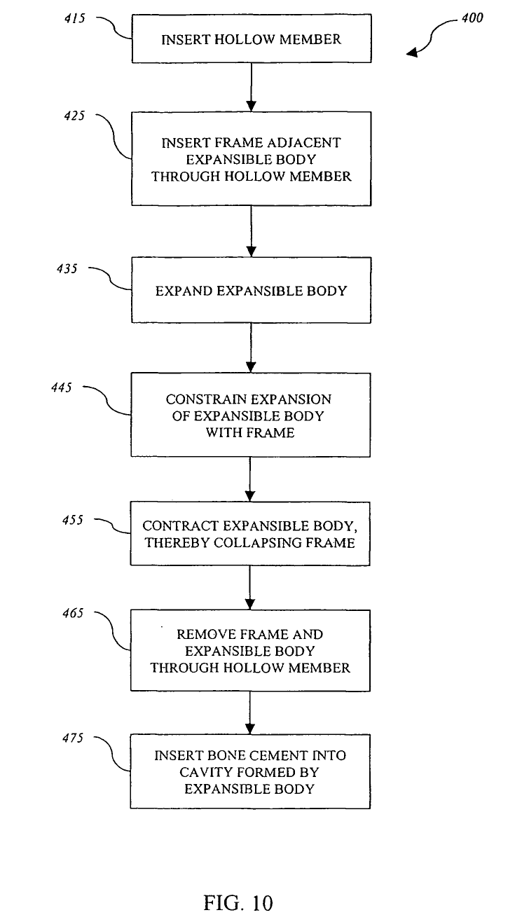

Referring now to FIG. 10, a flow chart of a method 400 according to one embodiment of the present invention is shown. The illustrative embodiment comprises percutaneously inserting a hollow member (such as the hollow member 20 described above) into a vertebral body of a vertebra comprising a vertical compression fracture condition as shown in box 415.

The method 400 further comprises inserting a frame disposed adjacent an expansible body (such as the frame 50 and the expansible body 80 described above) into the vertebral body through the hollow member as shown in box 425.

The method 400 further comprises expanding the expansible body once beyond a distal end of the hollow member and inside the vertebral body, as shown in box 435. For example, the expansible body may be expanded by movement of a flowable material into the expansible body through an aperture therein. A cavity is provided in the vertebral body as the expansible body is expanded.

The method 400 further comprises constraining expansion of the expansible body as it is expanded using the frame, as shown in box 445. For example, the frame may comprise a plurality of adjustable frame members oriented radially around the expansible body and along the axis of the hollow member as shown in FIG. 4. One or more of these adjustable frame members may then be adjusted by a user of the device to constrain growth of the expansible body to obtain the maximum possible height restoration of a vertebral body that is suffering from a vertical compression fracture condition.

The method 400 further comprises contracting the expansible body, thereby collapsing the frame, as shown in box 455. For example, the expansible body may be contracted by movement of a flowable material out of the expansible body through an aperture therein. In one embodiment, the expansible body may be contracted once a user has determined that an appropriate amount of height has been restored to the vertebral body suffering from the vertical compression fracture condition, or that a cavity of sufficient size and shape has been provided in the vertebral body.

The illustrative method 400 further comprises removing the frame and the expansible body through the hollow member, as shown in box 465. In other embodiments, one or both of the frame and the expansible body may be separable from the an elongated member used to insert them into the vertebral body, and may be left implanted in either an expanded or contracted state within the vertebral body while the elongated member is removed through the hollow member.

The method 400 finally comprises inserting a bone cement into the cavity formed by the expansible body, as shown in box 475. The bone cement may be inserted through the same hollow member through which the expansible body and frame were inserted, or in another embodiment may be inserted through a separate hollow member into the vertebral body, such as a contralateral hollow member. The bone cement, which remains in the cavity provided by the expansible body and the frame, may provide dimensional stability to the vertebral body after the expansible body and adjacent frame have been removed. Another surgical tool, such as a scope, may also be inserted into the cavity through the hollow member.

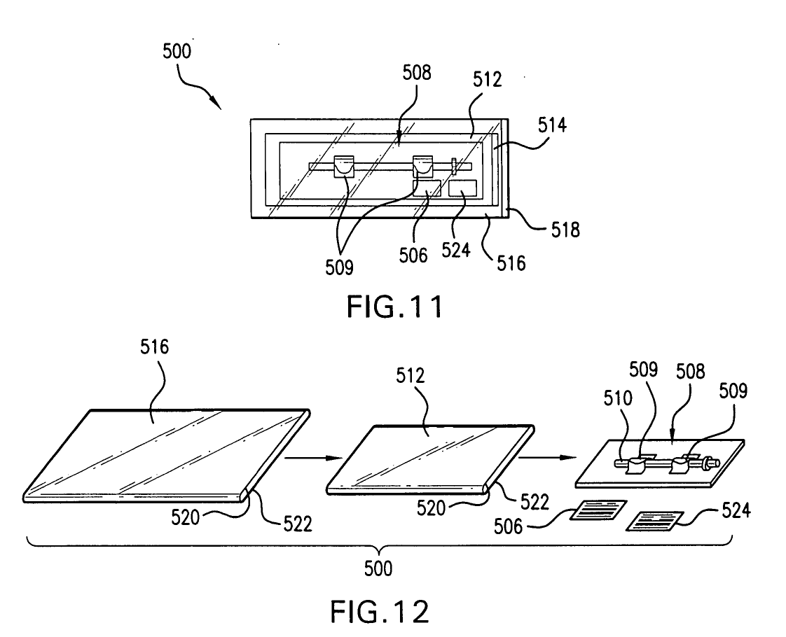

Referring now to FIGS. 11 and 12, a plan view and an exploded perspective view, respectively, of a sterile kit to store a cavity-forming tool according to one embodiment of the present invention is shown. A tool according to one embodiment of the present invention (such as the tool 210 described above) may be packaged in a sterile kit 500 as shown in FIGS. 11 and 12 prior to deployment in a bone or other tissue. In one such embodiment, the tool may comprise a single use tool.

As shown in FIGS. 11 and 12, the kit 500 comprises an interior tray 508. The tray 508 holds the particular cavity-forming tool (generically designated 510) in a lay-flat, straightened condition during sterilization and storage prior to its first use. The tray 508 can be formed from die cut cardboard or thermoformed plastic material. The tray 508 comprises one or more spaced apart tabs 509, which hold the tool 510 in the desired lay-flat, straightened condition.

The kit 500 comprises an inner wrap 512 that, in the embodiment shown, is peripherally sealed by heat or the like, to enclose the tray 508 from contact with the outside environment. One end of the inner wrap 512 comprises a conventional peal-away seal 514 (see FIG. 12), to provide quick access to the tray 508 upon use, which may occur in a sterile environment, such as within an operating room.

The kit 500 shown also comprises an outer wrap 516, which is also peripherally sealed by heat or the like, to enclose the inner wrap 512. One end of the outer wrap 516 comprises a conventional peal-away seal 518 (see FIG. 12), to provide access to the inner wrap 512, which can be removed from the outer wrap 516 in anticipation of imminent use of the tool 510, without compromising sterility of the tool 510 itself.

Both inner and outer wraps 512 and 516 (see FIG. 12) comprise a peripherally sealed top sheet 520 and bottom sheet 522. In the illustrated embodiment, the top sheet 520 is made of transparent plastic film, like polyethylene or MYLAR™ material, to allow visual identification of the contents of the kit 500. The bottom sheet 522 may be made from a material permeable to ethylene oxide sterilization gas, e.g., TYVEC™ plastic material (available from DuPont).

In the embodiment shown in FIGS. 11 and 12, the sterile kit 500 also carries a label or insert 506, which comprises the statement “For Single Patient Use Only” (or comparable language) to affirmatively caution against reuse of the contents of the kit 500. The label 506 also may affirmatively instruct against resterilization of the tool 510. The label 506 also may instruct the physician or user to dispose of the tool 510 and the entire contents of the kit 500 upon use in accordance with applicable biological waste procedures. The presence of the tool 510 packaged in the kit 500 verifies to the physician or user that the tool 510 is sterile and has not been subjected to prior use. The physician or user is thereby assured that the tool 510 meets established performance and sterility specifications, and will have the desired configuration when expanded for use.

The kit 500 also may comprise directions for use 524, which instruct the physician regarding the use of the tool 510 for creating a cavity in cancellous bone in the manners previously described. For example, the directions 524 instruct the physician to deploy, manipulate, and adjust the tool 510 inside bone to provide a cavity. The directions 524 can also instruct the physician to fill the cavity with a material, e.g., bone cement, allograft material, synthetic bone substitute, a medication, or a flowable material that sets to a hardened condition before, during, or after the tool 510 has provided the cavity.

One embodiment according to the present invention comprises a device comprising an expansible frame, a hollow member, and an elongate member. The expansible frame may comprise a shape memory material, such as a nickel-titanium alloy, and a cutting surface adapted to cut tissue. In one embodiment, the expansible frame may comprise a plurality of spaced-apart frame members axially oriented about the circumference of the tube. At least one of the plurality of spaced-apart frame members may comprise a cutting surface, such as a beveled edge, a deburred edge, a serrated edge, a blade tip, or a raised edge.

The hollow member may establish a path. The elongate member may comprise a distal end portion with an axis of rotation. At least a portion of the elongate member may be adapted to be maneuverable along the path established by the hollow member, and movably disposed within the hollow member.

The expansible frame may be disposed at the distal end portion of the elongate member, and may be rotatable about the axis of rotation of the elongate member. The expansible frame may be movable along and rotatable about the axis of the hollow member for cutting tissue in selected locations. In some embodiments, a liner material may be disposed inside or outside the expansible frame. In one such embodiment, the liner may comprise a sheath.

In one embodiment, the expansible frame may be expansible to a preset shape and preset dimensions. The shape memory material may comprise a radiopaque material.

Further embodiments may comprise an expansible body adapted to be disposed inside the expansible frame. The expansible body may be adapted to compress and maintain tissue in selected directions and to selected dimensions. The expansible body may be attachable to the distal end of the tube. In one embodiment comprising an expansible body, the expansible frame may be capable of being expanded independently of the expansible body. In another such embodiment, when the expansible body is partially expanded, the expanded expansible body may exert pressure on the expansible frame to provide pressure-assisted cutting. The expansible frame and the expansible body may be packaged together in a single kit.

One device according to the present invention may further comprise a means for introducing a therapeutic material through the hollow member.

Another embodiment according to the present invention comprises a hollow member, an expansible frame, and an expansible body adapted to be disposed inside the expansible frame. The hollow member may comprise an axis establishing a path.

The expansible frame may be expansible to a preset shape and preset dimensions. The expansible frame may comprise a tube adapted to be deployed by movement within and along the axis of the hollow member. The tube may comprise a distal end comprising a shape memory material (such as a nickel-titanium alloy) and a plurality of spaced-apart frame members axially oriented about the circumference of the tube. The shape memory material may comprise a radiopaque material. At least one of the frame members may comprise a cutting surface. The expansible frame may be movable along and rotatable about the axis of the hollow member for cutting tissue in selected locations.

The expansible body may be adapted to be disposed inside the expansible frame. The expansible body may be adapted to compress and maintain tissue in selected directions and to selected dimensions. In one embodiment, when the expansible body is partially expanded, the expanded expansible body may exert pressure on the expansible frame to provide pressure-assisted cutting. In one embodiment, the expansible frame may be capable of being expanded independently of the expansible body.

One method according to the present invention comprises providing a device comprising (i) a hollow member having an axis and a distal end, and (ii) an expansible frame comprising a shape memory material (such as a nickel-titanium alloy) and a cutting surface, the expansible frame disposed at the distal end of a tube. For example, the expansible frame may comprise a plurality of spaced-apart frame members axially oriented about the circumference of the tube. At least one of the frame members may comprise a cutting surface. In one embodiment, the device may include an expansible body disposed inside the expansible frame. In such an embodiment, the expansible body may be inflated to compress the tissue in selected directions and to selected dimensions.

The hollow member may be inserted to establish a path along the axis of the hollow member. The device may then be deployed by moving the tube within and along the axis of the hollow member. The expansible frame may be moved beyond the distal end of the hollow member and along and about the axis of the hollow member to cut tissue in selected locations. The expansible frame may be expanded to a preset shape and preset dimensions, for example by superelastic motion of the shape memory material.

In another embodiment, one of the devices may be deployed to each of two sides of a vertebral body. In such an embodiment, after inflating an expansible body, one of the devices may be withdrawn from one side of the vertebral body, and a therapeutic material may be introduced into a space created on the side of the vertebral body from which the one of the devices was withdrawn. The remaining device may then be withdrawn from the other side of the vertebral body, and the therapeutic material may be introduced into a space created on the side of the vertebral body from which the remaining device is withdrawn.

In an embodiment where the shape memory material comprises a radiopaque material, the movement and positioning of the radiopaque material may be visualized to observe movement and positioning of the expansible frame.

The foregoing description of embodiments of the invention has been presented only for the purpose of illustration and description and is not intended to be exhaustive or to limit the invention to the precise forms disclosed. Numerous modifications and adaptations thereof will be apparent to those skilled in the art without departing from the spirit and scope of the present invention.

Furthermore, where methods and steps described above indicate certain events occurring in certain orders, those of ordinary skill in the art having the benefit of this disclosure would recognize that the ordering of certain steps may be modified and that such modifications are in accordance with the variations of the invention. Additionally, certain of the steps may be performed concurrently in a parallel process when possible, as well as performed sequentially as described above. Thus, the breadth and scope of the invention should not be limited by any of the above-described embodiments, but should be defined only in accordance with the following claims and their equivalents.

What is claimed is:

1. A system comprising:
an expansible body configured to be deployed within a treatment area; and
a frame comprising a plurality of frame members disposed adjacent the expansible body, the frame configured to constrain expansion of the expansible body and to be maneuverable along a path established by a hollow member.

2. The system of claim 1, wherein at least a portion of the frame is disposed within the expansible body.

3. The system of claim 1, wherein at least a portion of the expansible body is disposed within the frame.

4. The system of claim 1, wherein the expansible body is configured to be expanded by movement of a flowable material into the expansible body.

5. The system of claim 1, wherein the expansible body is configured to be contracted by movement of a flowable material out of the expansible body.

6. The system of claim 1, wherein the frame is configured to collapse as the expansible body contracts.

7. The system of claim 1, wherein the frame is configured to be at least one of removed from the treatment area and inserted into the treatment area along the path established by the hollow member.

8. The system of claim 1, wherein at least a portion of the frame is adjustable to control a dimension of the expansible body.

9. The system of claim 1, wherein the plurality of frame members are individually adjustable to control at least one dimension of the expansible body.

10. The device of claim 1, wherein the plurality of frame members are axially oriented about the expansible body.

11. The system of claim 1, further comprising:
an elongate member comprising a distal end portion, wherein at least the distal end portion of the elongate member is configured to be movably disposed along the path established by the hollow member, and wherein at least one of the frame and the expansible body is disposed at the distal end portion of the elongate member.

12. The system of claim 1, wherein the frame comprises a surface configured to contact and shear a tissue.

13. The system of claim 1, wherein at least a portion of the frame is coupled to the expansible body.

14. The system of claim 1, wherein the frame comprises a structural member encased within a tubular member adjacent the expansible body.

15. The system of claim 1, further comprising:
a sheath surrounding the frame and the expansible body.

16. The system of claim 15, wherein the sheath comprises a substantially non-compliant material.

17. The system of claim 15, wherein the sheath comprises a rupture-resistant material.

18. The system of claim 15, wherein the frame is disposed between the sheath and the expansible body.

19. The system of claim 18, wherein the sheath is coupled to the expansible body.

20. The system of claim 1, further comprising:
a spring member coupled to the frame, the spring member configured to at least one of expand the frame and contract the frame.

21. The system of claim 1, further comprising:
a screw member coupled to the frame, the screw member configured to at least one of expand the frame and contract the frame.

22. The system of claim 1, wherein the frame is configured to be implanted within the treatment area.

23. The system of claim 1, wherein the expansible body is configured to be expanded from a first dimension to a second dimension when extended to a point beyond a distal end of the hollow member.

24. The system of claim 23, wherein the expansible body is configured to be contracted from the second dimension to the first dimension when brought within the distal end of the hollow member from the point beyond the distal end of the hollow member.

25. The system of claim 1, wherein at least a portion of the frame comprises a shape memory material.

26. A method comprising:
inserting a frame comprising a plurality of frame members disposed adjacent an expansible body into a treatment area along a path established by a hollow member;
expanding the expansible body in the treatment area; and
constraining expansion of the expansible body with the frame.

27. The method of claim 26, further comprising:
contracting the expansible body, thereby collapsing the frame; and
removing the frame along the path established by the hollow member.

28. The method of claim 26, further comprising:
adjusting the frame to control a dimension of the expansible body.

29. The method of claim 28, wherein adjusting the frame to control a dimension of the expansible body comprises individually adjusting at least one of the plurality of frame members to control a dimension of the expansible body.

30. The method of claim 26, wherein the frame comprises a surface configured to directly contact and shear a tissue, and further comprising:
contacting the tissue with the surface.

31. The method of claim 26, further comprising:
inserting a filler material into a cavity formed by at least one of the expansible body and the frame.

32. The method of claim 26, further comprising:
at least one of expanding and contracting the frame with a spring member.

33. The method of claim 26, further comprising:
at least one of expanding and contracting the frame with a screw member.

34. The method of claim 26, further comprising:
applying a therapeutic material to at least a portion of the frame.

35. The method of claim 34, further comprising:
introducing the therapeutic material to a tissue.

36. A device comprising:
an expansible frame comprising a shape memory material and a cutting surface configured to cut tissue; and
an elongate member comprising a distal end portion with an axis of rotation, at least a portion of the elongate member adapted to be maneuverable along a path established by a hollow member,
the expansible frame being disposed at the distal end portion of the elongate member and rotatable about the axis of rotation.

37. The device of claim 36, wherein the expansible frame is expansible to at least one of a preset shape and a preset dimension.

38. The device of claim 36, wherein the shape memory material comprises a nickel-titanium alloy.

39. The device of claim 36, wherein the shape memory material comprises a radiopaque material.

40. The device of claim 36, wherein the elongate member comprises a tube, and wherein the expansible frame comprises a plurality of spaced-apart frame members axially oriented about a circumference of the tubular elongate member.

41. The device of claim 40, wherein at least one of the plurality of spaced-apart frame members comprises the cutting surface.

42. The device of claim 41, wherein the at least one of the plurality of spaced-apart frame members comprises a beveled edge.

43. The device of claim 36, wherein the expansible frame is movable along and rotatable about an axis of the hollow member for cutting tissue in selected locations.

44. The device of claim 36, further comprising an expansible body configured to be disposed inside the expansible frame.

45. The device of claim 44, wherein the expansible body is adapted to compress and maintain tissue in selected directions and to selected dimensions.

46. The device of claim 44, wherein the expansible body is attachable to the distal end of the elongate member.

47. The device of claim 44, wherein the expansible frame is configured to be expanded independently of the expansible body.

48. The device of claim 44, wherein when the expansible body is partially expanded, the expanded expansible body exerts pressure on the expansible frame to provide pressure-assisted cutting.

49. The device of claim 36, further comprising a means for introducing a therapeutic material through the hollow member.

50. The device of claim 36, further comprising a liner material disposed inside the expansible frame.

51. The device of claim 44, wherein the expansible frame and the expansible body are packaged together in a single kit.

52. A device comprising:
an expansible frame configured to be expanded to a preset shape and a preset dimension, the expansible frame comprising a tube adapted to be deployed by movement within and along an axis of a hollow member, the tube having a distal end comprising a shape memory material and a plurality of spaced-apart frame members axially oriented about a longitudinal axis of the expandable frame, at least one of the frame members having a cutting surface, the expansible frame movable along and rotatable about the axis of the hollow member for cutting tissue in selected locations; and
an expansible body adapted to be disposed inside the expansible frame.

53. The device of claim 52, further comprising:
the hollow member having the axis establishing a path.

54. The device of claim 52, wherein the shape memory material comprises a nickel-titanium alloy.

55. The device of claim 52, wherein the shape memory material comprises a radiopaque material.

56. The device of claim 52, wherein the expansible body is adapted to compress and maintain tissue in selected directions and to selected dimensions.

57. The device of claim 52 wherein the expansible frame is capable of being expanded independently of the expansible body.

58. The device of claim 52, wherein when the expansible body is partially expanded, the expanded expansible body exerts pressure on the expansible frame to provide pressure-assisted cutting.

59. A method comprising:
providing a device comprising (i) a hollow member having an axis and a distal end and (ii) an expansible frame comprising a shape memory material and a cutting surface, the expansible frame disposed at the distal end of a tube;
inserting the hollow member to establish a path along the axis of the hollow member;
deploying the device by moving the tube within and along the axis of the hollow member;
moving the expansible frame beyond the distal end of the hollow member and along and about the axis of the hollow member to cut tissue in selected locations; and
expanding the expansible frame.

60. The method of claim 59, wherein expanding the expansible frame comprises expanding the expansible frame to a preset shape and a preset dimension.

61. The method of claim 59, further comprising expanding the expansible frame to a preset shape and a preset dimension by superelastic motion of the shape memory material.

62. The device of claim 59, wherein the shape memory material comprises a nickel-titanium alloy.

63. The method of claim 59, wherein the expansible frame comprises a plurality of spaced-apart frame members axially oriented about the circumference of the tube, and wherein at least one of the frame members comprises a cutting surface.

64. The method of claim 59, the device including an expansible body disposed inside the expansible frame, the method further comprising inflating the expansible body to compress the tissue in selected directions and to selected dimensions.

65. The method of claim 64, further comprising:
deploying one of the devices to each of two sides of a vertebral body;
after inflating the expansible body, withdrawing one of the devices from one side of the vertebral body;
introducing a therapeutic material into a space created on the side of the vertebral body from which the one of the devices is withdrawn;
withdrawing the remaining device from the other side of the vertebral body; and
introducing the therapeutic material into a space created on the side of the vertebral body from which the remaining device is withdrawn.

66. The method of claim 59, the shape memory material comprising a radiopaque material, the method comprising visualizing the movement and positioning of the radiopaque material to observe movement and positioning of the expansible frame.
